(19) United States
(12) Patent Application Publication
(10) Pub. No.: US 2015/0335466 A1
(43) Pub. Date: Nov. 26, 2015

(54) EAR COOLING DEVICE

(71) Applicant: Christoph SCHÖGGLER, (US)

(72) Inventor: Christoph SCHÖGGLER, Wundschuh (AT)

(21) Appl. No.: 14/435,575

(22) PCT Filed: Oct. 15, 2012
(86) PCT No.: PCT/AT2012/050163

§ 371 Date: Jul. 14, 2015

(51) Int. Cl. A61F 7/00

(52) U.S. Cl. CPC A61F 7/007; A61F 2007/0005; A61F 2007/0075; A61F 2007/0095

(57) ABSTRACT

A mobile device (1) that can be worn on the ear for cooling the outer ear in order to support the healing process in the case of ear canal inflammation, said device comprising a ear plug (2) which can be placed against an ear canal, particularly an ear canal of a human being, and can preferably be at least partially introduced into the car canal, and a cooling device by means of which the ear plug (2) and/or the air in the ear canal can be cooled. The cooling device is designed without liquid heat transfer means and comprises a Peltier element, the inner side (31) of which is connected to the ear plug (2) in order to cool said plug and/or the air in the ear canal, and via the outer side (32) of which heat can be dissipated.

The invention relates to a mobile device that can be worn on the ear for cooling the outer ear in order to support the healing process in the case of ear canal inflammations, comprising an ear plug which can be placed against an ear canal, particularly an ear canal of a human being, and can preferably be at least partially inserted into said ear canal, and a cooling device by means of which the ear plug and/or air located in the ear canal can be cooled.

In treating different ailments of a human ear, it can be necessary to cool or heat the ear. For this purpose, liquids, particularly water, are typically used that are brought to a predetermined temperature. In this context, a device has become known from JP 2006230761 A, for example, in which device water from a water tank is brought to a preset temperature by means of Peltier elements, in order to heat the back side of a human ear.

In addition to applying a liquid to a human ear in order to heal an ailment, a temperature treatment of an ear may also be provided for other reasons, for example, in order to test the efficacy of and check for potential pathological changes to the human vestibular system. In this regard, a device has become known from DE 23 23 444 A1 in which a Peltier element is connected on one side to a metal plug of copper, which plug can be inserted into a human ear. On the opposite side, the Peltier element is connected to a cooling device which comprises a water circuit. By controlling the Peltier element, it is possible to bring the metal plug to a specific temperature and to thus stimulate the vestibular system. On the other side, heat is dissipated by the water. The device can be portable if the water cooling is designed as a circulation cooling system.

For patients with an ear canal inflammation (otitis externa), a cooling of the outer ear is specifically used. Otitis externa is a bacterial, fungus-induced inflammation of the outer ear canal, as a further consequence of which the eardrum can also be affected. In addition to a medicinal therapy, a cooling and drying of the outer ear is also particularly recommended by doctors, in order to achieve a quick alleviation of pain and a rapid healing.

Ear inflammations occur relatively frequently and, accordingly, must also often be treated. No suitable devices are presently available for the recommended cooling and, where necessary, drying of an ear by the patient himself/herself. The known devices are mostly elaborately designed and therefore not portable. This means that a patient must always visit a hospital or other medical institution for treatment.

The object of the invention is to specify a device by means of which the outer ear canal of an ear can be cooled with low instrument-related cost, and wherein at the same time the ear is protected against undesired moisture and air draft.

The object is attained if, for a device of the type named at the outset, the cooling device is embodied without a liquid means of heat transport and comprises a Peltier element, the inner side of which is connected to the ear plug in order to cool said ear plug and/or air located in the ear canal, and via the outer side of which heat can be dissipated.

In contrast to the prior art, no liquid means of heat transport is provided according to the invention; rather, a preferably direct dissipation of heat to the air occurs through radiation on the outer side of the Peltier element. The knowledge is thereby utilized that, for a cooling of the outer ear in order to support a healing process in the case of ear canal inflammations, only a slight temperature reduction of a few degrees is necessary. Larger temperature reductions are also not at all desirable, since the sense of balance can thus be disrupted and nausea can also be induced in the patient. Because a slight cooling is already fully sufficient for the intended healing purposes, in particular the treatment of ear canal inflammations, an elaborately constructed water cooling system can be omitted in the operation of the Peltier element, for which reason the device according to the invention can be realized with little means or with relatively simple instruments overall.

For the desired cooling, the ear plug can be placed against the ear canal, which may be sufficient. In most cases, it is advantageous that the ear plug can be at least partially inserted into the ear canal in order to efficiently activate the cooling effect at the point of inflammation. Through the careful insertion into the ear canal, a region of the ear plug at the end thereof can be brought into contact with an inflamed point, without the inflamed point being irritated.

In addition, the ear plug provided ensures that, during the treatment, the ear canal can be at least mostly closed off from the surrounding environment, or that it hardly has any contact with the surrounding environment, whereby an ingress of undesired moisture or an unwanted air draft can be avoided in the ear canal.

Essentially, metals or plastics are suitable as materials for the ear plug. As metals, aluminum or titanium, as well as alloys with these metals, can be used as main components, for example. However, an embodiment of the ear plug from a plastic, particularly a polymer such as polyoxymethylene, is preferred. The ear plug is thereby preferably partially or completely formed from an elastic plastic. This makes it possible for the ear plug to be fitted to the ear during insertion, so that a desired closure of the ear canal is ensured during the subsequent treatment.

The ear plug can be embodied with a removable attachment. The attachment, which is located at an end of the ear plug facing the ear, can then be removed after use or after a treatment and replaced by a new attachment. As a result, a hygienically clean attachment can he used for each individual treatment.

It can also be provided that the ear plug is at least partially coated. A coating can be provided for various reasons. A coating with silicone is, for example, expedient if the ear plug is composed of a metal. The external coating with silicone then gives the ear plug a desired elasticity in the outer region, so that said ear plug can be inserted into the ear or the ear canal and fitted to the ear canal, despite the inherently rigid fabrication from a metal. However, a coating can also be provided in order to reinforce an antibacterial effect. For this purpose, a coating of silver is used, for example. Within the scope of the invention, it is also possible that a coating is used which releases silver ions gradually over time and thus has a lasting antibacterial effect. It can thereby be sufficient that the ear plug is only provided with the antibacterial coating in certain regions.

The ear plug is essentially shaped such that it forms on a first end, which can be inserted into the ear canal, a sealed or continuous surface. The ear plug itself is cooled by the Peltier element, so that the desired cooling effect is achieved. However, it is also possible that the ear plug comprises an opening at the first end. In this alternative, air located in the ear canal can enter into the ear plug and be cooled there. The low circulation is then sufficient to cool the air located in the ear canal and thus ultimately also the ear canal,

The Peltier element can in principle be arranged in any desired position, provided that a required effective connection to the ear plug is ensured. Preferably, the Peltier element is arranged in a region of the ear plug at the end thereof and arranged inside of said ear plug. A simple and compact design is thus ensured. In particular, it is thus also possible for the ear plug to surround the Peltier element to a large extent, so that only the outer side of the Peltier element, which is provided for the heat dissipation, is accessible. This outer side can, for example, also be embodied with an element that intensifies the cooling effect, such as a ribbed small metal leaf that is connected to the Peltier element and via the surface of which the heat is dissipated. It is thereby advantageous that the device can not only be easily handled by the patient, but that it can also hardly be damaged by the patient.

For the operation of the Peltier element, a first line or a cable is expediently provided with which the Peltier element is connected to the power supply. Normally, the cable comprises at one end a plug by means of which it can he connected to a power supply source. It is particularly advantageous that the cable comprises a plug at its end, which plug can be plugged into a mobile telephone. Thus, a power supply for the device according to the invention can be ensured in transit in a simple manner.

It is normally also expedient that a supply and control unit is provided which is connected to the Peltier element and by means of which the Peltier element can be controlled. Although the supply and control unit can be provided in any desired position, it is expediently arranged between the power source and the device according to the invention. Cooling sequences can thus be structured in any desired manner. For example, it is possible to set an interval cooling or also a pulsating cooling. In addition, the supply and control unit allows the presetting of specific maximum values for the temperature, so that a temperature range within which the treatment is to occur can be set. Thus, an undesired exceeding of a minimum temperature, below which nausea might result due to a disruption of the sense of balance, can be avoided, for example. Here, a display for indicating the temperature can also be provided. In order to output various different parameters, an acoustic output device can alternatively or additionally be integrated.

The supply and control unit can comprise a rechargeable battery and an operating element for adjusting the temperature and a rectifier for generating direct current.

If a power supply is already ensured per se by a mobile telephone, the supply and control unit can also be integrated in the ear plug. In this case, a set exists for cooling the ear canal of an ear, which set is composed of a device according to the invention together with a cable and mobile telephone. The supply and control device then ensures that a necessary direct current for operating the Peltier element is supplied. This current is supplied by the mobile telephone. The advantage of this alternative is that the cooling of the ear plug can also be adjusted via the control panel of the mobile telephone or via a corresponding application.

If a temperature is to be controlled precisely, it is expedient if a temperature sensor is arranged in the ear plug. This temperature sensor is thereby preferably arranged in the region of the first end of the ear plug, which end faces the ear canal.

Particularly for the noted combination of the device according to the invention with a mobile telephone, it is expedient that a speaker is integrated in the ear plug. Listening to music or carrying on a telephone conversation is thus possible while a patient is being treated.

The ear plug is basically embodied such that it already sits firmly in the ear on its own. However, it can also be provided that a mount connects to the ear plug, by means of which mount the device can be attached to the ear. An additional grip or a particularly firm fastening to the ear can thus be achieved. As a secondary aspect, it thereby also results that, if necessary, the mount can also be designed such that it can be cooled, so that outer regions of the ear can likewise be cooled.

For treatments that additionally also require the delivery of drugs and/or disinfectants, it is advantageous to also provide a line for the delivery of a drug or disinfectant, which line ends at an outer surface of the ear plug. In addition to the cooling, this allows the delivery of substances which promote healing during treatment.

Additional features, advantages and effects of the invention follow from the exemplary embodiments described below. The drawings which are thereby referenced show the following:

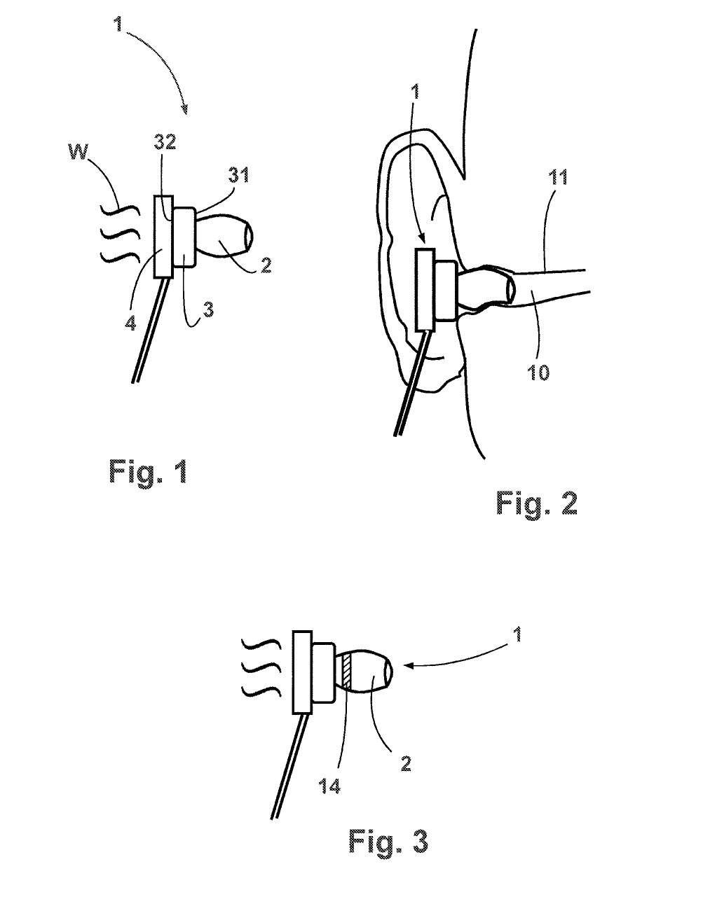

FIG. 1 Device according to the invention;

FIG. 2 The arrangement of a device according to the invention in an ear or ear canal;

FIG. 3 A further exemplary embodiment of a device according to the invention;

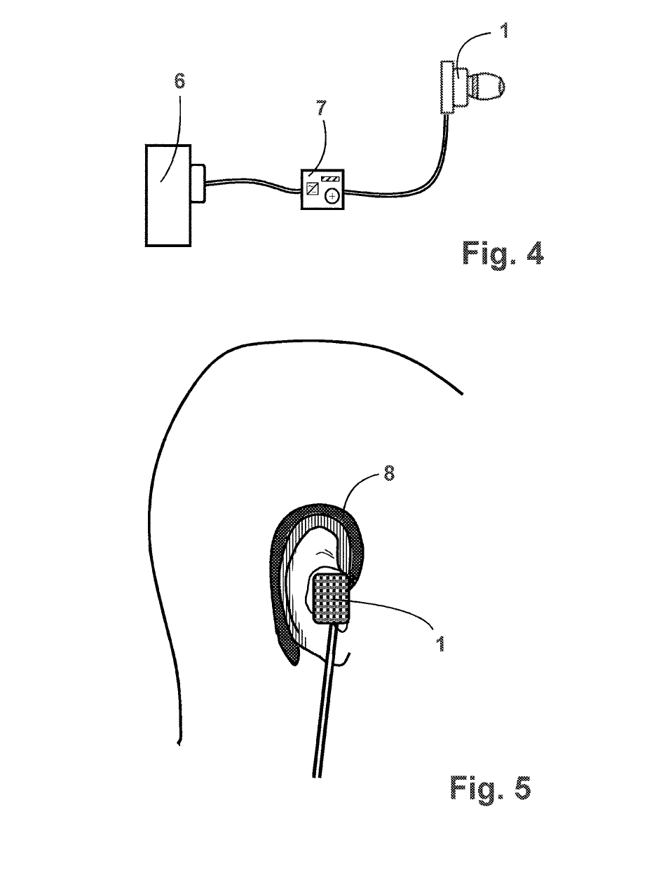

FIG. 4 A device according to the invention with a supply and control unit;

FIG. 5 An additional alternative of a device according to the invention;

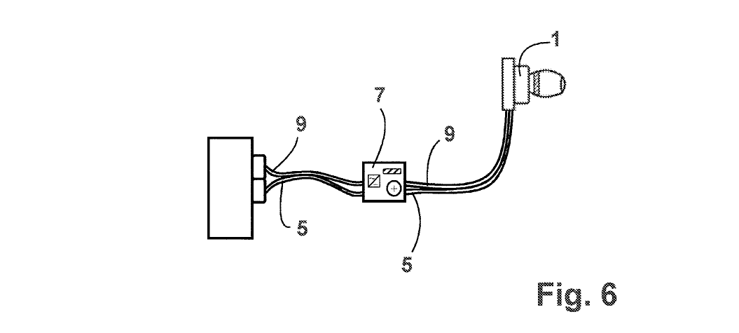

FIG. 6 A device according to the invention with multiple lines;

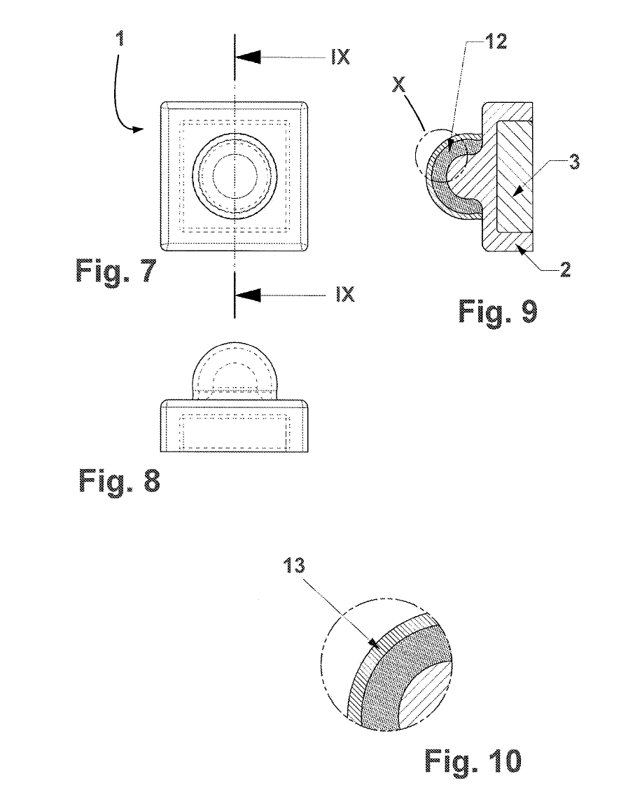

FIG. 7 A device according to the invention with an attachment in a top view;

FIG. 8 The device according to FIG. 7 in a front-face view;

FIG. 9 The device according to FIG. 7 in cross section along the line IX-IX in FIG. 7;

FIG. 10 A detailed view of the device according to FIG. 9;

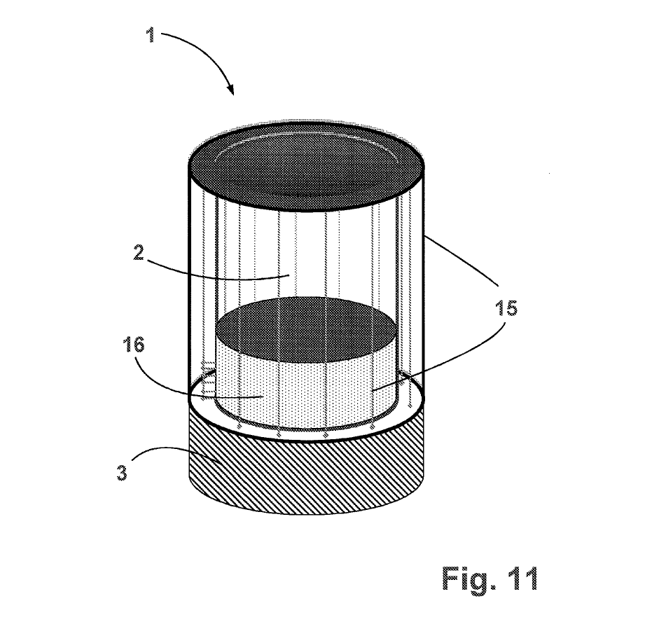

FIG. 11 An additional alternative of a device according to the invention.

A device 1 according to the invention is illustrated in FIG. 1. The device 1 comprises an ear plug 2, as well as a Peltier element 3 and a cooling body 4. On an inner side 31, the Peltier element 3 is in contact with the ear plug 2. On an outer side 32, the Peltier element 3 is in contact with a cooling body 4. Although the cooling body 4 can be provided, this is not necessary, In addition, a line 5 is provided which runs through the cooling body 4 and via which current for operating the Peltier element 3 is fed to the same.

The ear plug 2 can be formed from any desired material. Typically, the ear plug 2 is formed from a metal such as aluminum or titanium, or from alloys of these metals. In particular, titanium or titanium alloys can be provided, since these are biocompatible and do not induce allergic reactions. However, it is particularly preferred that the ear plug 2 is composed of an elastic plastic. For example, polyoxymethylene can be used for this purpose. The ear plug 2 can then be elastically deformed with slight pressure and can be fitted to an ear canal 10 or an ear canal wall 11, as this is illustrated schematically in FIG. 2. Generally, it is advantageous that the ear plug 2 is composed of a pliable and skin-friendly material. In particular in case of the use of metals it can be advantageous to apply an additional coating of an elastic material, for example, silicone, so that the desired elasticity, and therefore pliability, is ensured in at least the outer region of the ear plug 2.

As can be seen in FIGS. 1 and 2, a device 1 according to the invention does not require any liquid cooling medium or liquid means of heat transport whatsoever. The device 1 can thus be manufactured compactly and is mobile and, in particular, also easy to transport. This allows a patient to use the device 1 at any time, provided that a power supply is present. Significant flexibility is thus ensured in the case of acute pain. At the same time, with the elastic ear plug 2, an essentially sealed closure of the ear canal 10 is ensured, so that no external air draft in the ear occurs during the treatment of acute pain.

As illustrated schematically in FIG. 3, a device 1 can also comprise a speaker 14, so that listening to music or, if necessary, carrying on a telephone conversation is possible during a treatment.

In FIG. 4, a device 1 is illustrated which is connected to a mobile telephone 6 via a supply and control unit 7. In this case, the ear plug 2 expediently comprises a temperature sensor not shown which, for example, is arranged in a region of a first end of the ear plug 2 facing the ear canal 10, Via a first line 5 or a cable, the Peltier element 3 is connected to the supply and control unit 7. The same applies to the integrated temperature sensor not shown. With the supply and control unit 7, a temperature can on the one hand be set precisely. Preferably, a temperature interval is set, within which the Peltier element 3 is to operate. The supply and control device 7 is fed with current via the mobile telephone 6, typically an alternating current. Therefore, a rectifier is additionally provided in the supply and control until 7, in order to generate the direct current for operating the Peltier element 3.

It can also be provided that the first line 5 or the cable is embodied with a plug at its end, so that the device 1 can be directly connected to a mobile telephone 6 or, if necessary, another terminal device, such as an MP3 player or a laptop, via a USB port or another standardized interface, for example. In this case, it is expedient that an adjustment of the temperature, or an operation of the device 1 generally, is carried out via the mobile telephone 6, particularly an application running on the mobile telephone 6. Where necessary, in particular for generating direct current, the supply and control unit 7 can be integrated in the ear plug 2, as will be further explained below.

In FIG. 5, an alternative of a device according to the invention is illustrated which also comprises a mount 8. The mount 8 can have two functions. On the one hand, the mount 8 is used to fasten the device 1 to a human ear more effectively. On the other hand, it can also be provided that the mount 8 is cooled as well, whereby outer regions of the ear can also be treated, for example, in the case of swelling in the outer region of the ear.

In FIG. 6, a device 1 according to the invention is illustrated which comprises in addition to the first line 5 or the cable a second line 9 which is embodied in a hollow manner and is used to deliver a drug and/or disinfectant. For this purpose, a separate tank is provided through which small amounts of a drug can be delivered.

The second line 9 can, however, also be embodied in a different manner, namely, as a cable similar to the first line 9. In this case, the power supply can be arranged in the first line 5, while an audio signal can he transmitted in the second line 9.

A further alternative for a device 1 according to the invention is illustrated in FIGS. 7 through 10. The device 1 once again comprises an ear plug 2 which is formed from an elastic plastic. In this case, the ear plug 2 is printed from a polymer using a 3D printer. At one end, the ear plug 2 comprises a recess into which the Peltier element 3 is inserted. In addition to the Peltier element 3, which is only illustrated in a highly schematic matter, a supply and control unit 7 can also be embedded in the ear plug 2. The ear plug 2, which is elastically deformable, thus comprises all components necessary for cooling the ear, except for a power supply. It is thereby advantageous that, as a result of the envelopment of the electronic components by the ear plug 2, damage to the device 1 by a patient is hardly possible, even if the device 1 is used repeatedly. This damage protection, and thus a durability, of the product can be improved even further if at the end facing away from the ear the ear plug 2 is closed off by a cooling body 4 which not only better dissipates heat from the Peltier element 3, but also constitutes a mechanical protection. Suitable for this is a small metal plate or a metal plate, which are preferably embodied with ribs.

Furthermore, the cooling body 4, which is not absolutely necessary per se, can also be shaped in an aesthetically attractive manner and/or be fitted to an outer side of the ear.

In an alternative, the ear plug 2 according to FIGS. 7 through 10 can also be formed from a metal or an alloy, particularly aluminum or an aluminum alloy. In this case, an adequate conductivity of heat away from Peltier element 3 is achieved.

In the alternative embodiment according to FIGS. 7 through 10, an attachment 12 can be provided which can be attached to the ear plug 2 at the end thereof, namely, regardless of the material of which the ear plug 2 is composed. For this purpose, the attachment 12 is advantageously formed from an elastic material, so that the projection on the ear plug 2 at the end thereof can be easily covered with the attachment 12. The attachment 12 is easily interchangeable and can be replaced by a new attachment if the device 1 is used by different people, for example. It is also possible that in addition to universally usable attachments 12, special attachments 12 for a left ear and a right ear are provided. Although the attachment 12 is preferably pushed on, other types of connection are also possible, for example, screwing on, sliding on, or hook-and-loop fastening.

Moreover, other components can also be provided in the ear plug 2. In one alternative, a small tank is provided in the ear plug, which tank contains a drug and/or disinfectant. The tank can thereby be embodied such that a drug is released when pressure is applied. The tank can also be provided in an attachment 12. Different drugs can then be delivered using different attachments 12.

It is also possible, as illustrated particularly in FIG. 10, that the ear plug 2 is at least partially provided with a coating. For example, the attachment 12 can be coated. Wearing comfort can be improved with a coating, for example, with antiallergenic coatings. However, it is also possible to provide antibacterial coatings. Another coating option is to use a specific active substance that is gradually released during the treatment.

An additional alternative for a device 1 according to the invention is illustrated in FIG. 11. In this case, the Peltier element 3 is again directly connected to a flexible ear plug 2. The ear plug 2 is also composed of a polymer material through which, however, a plurality of heat-conducting wires 15, particularly metal wires, pass, which wires are connected to the Peltier element 3 by a metal block 16. A heat transport is optimized by the heat-conducting wires 15, so that a particularly rapid cooling is achieved where necessary. In this context, it is also conceivable to use a polymer that is enriched with a metallic powder. In this case, it may he possible to omit the heat conducting wires in some circumstances.

Although the aforementioned exemplary embodiments have primarily been explained in relation to a device 1 for use in treating a human being, the device 1 can of course also he used on animals. In this case, it is advisable that the device 1 additionally comprises a collar or the like, by means of which the device can be fastened to the body of an animal that is to be treated.

In other alternatives, the ear plug 2 can be embodied according to one of the exemplary embodiments explained above, while additionally comprising a receptacle for a battery, so that the device 1 can be operated without additional devices. Furthermore, the ear plug 2 can, particularly in those regions which are inserted into the ear, be embodied with a textured surface, for example, with grooves, perforations, nubs or the like, to enable the application of topical liquid or cream-based substances in combination with therapy.

The device 1 according to the invention can, with corresponding adaptation, also be embodied for insertion into a different bodily orifice, such as the nose, vagina or anus, wherein the ear plug 2 then acts, with otherwise unaltered features, as a correspondingly adapted nose plug, vaginal plug or anal plug and can be worn on the body and/or in the bodily orifice.

1. Mobile device (1) that can be worn on the ear for cooling the outer ear in order to support the healing process in the case of ear canal inflammations, comprising an ear plug (2) which can be placed against an ear canal (10), particularly an ear canal (10) of a human being, and can preferably be at least partially inserted into said ear canal, and a cooling device by means of which the ear plug (2) and/or air located in the ear canal (10) can be cooled, characterized in that the cooling device is embodied without a liquid means of heat transport and comprises a Peltier element (3), the inner side (31) of which is connected to the ear plug (2) in order to cool said ear plug and/or air located in the ear canal (10), and via the outer side (32) of which heat can be dissipated.

2. Device (1) according to claim 1, characterized in that the ear plug (2) is partially or completely formed from an elastic plastic.

3. Device (1) according to claim 1, characterized in that the ear plug (2) is embodied with a removable attachment (12).

4. Device (1) according to claim 1, characterized in that the ear plug (2) is at least partially coated.

5. Device (1) according to claim 1, characterized in that the ear plug (2) comprises an opening at a first end, which end can be inserted into the ear canal.

6. Device (1) according to claim 1, characterized in that the Peltier element (3) is arranged in a region of the ear plug (2) at the end thereof and arranged inside of said ear plug.

7. Device (1) according to claim 1, characterized in that a first line (5) or a cable is provided to which the Peltier element (3) is connected in order to supply power to the same.

8. Device (1) according to claim 7, characterized in that the cable comprises a plug at its end, which plug can he plugged into a mobile telephone (6).

9. Device (1) according to claim 1, characterized in that a supply and control unit (7) is provided which is connected to the Peltier element (3) and by means of which the Peltier element (3) can be controlled.

10. Device (1) according to claim 9, characterized in that the supply and control unit (7) comprises a rechargeable battery and an operating element for adjusting the temperature and a rectifier for generating direct current.

11. Device (1) according to claim 1, characterized in that a temperature sensor is arranged in the ear plug (2).

12. Device (1) according to claim 1, characterized in that a speaker (14) is arranged in the ear plug (2).

13. Device (1) according to claim 1, characterized in that a mount (8) connects to the ear plug (2), by means of which mount the device can be attached to the ear.

14. Device (1) according to claim 1, characterized in that a second line (9) for the delivery of a drug or disinfectant is provided, which line ends at an outer surface of the ear plug (2).
